(19) United States
(12) Patent Application Publication
(10) Pub. No.: US 2004/0009499 A1
(43) Pub. Date: Jan. 15, 2004

(54) Method and system for allergy analysis

(75) Inventors: Hiroyuki Tomita, Tachikawa (JP); Toshiro Saito, Hatoyama (JP); Masatoshi Narahara, Sayama (JP); Hirokazu Kato, Hatoyama (JP); Emiko KIkkawa, Kokubunji (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/352,901

(22) Filed: Jan. 29, 2003

(30) Foreign Application Priority Data
Jul. 12, 2002 (JP) 2002--204785

(51) Int. Cl. C12Q 1/68; G06F 19/00; G01N 33/48; G01N 33/50

(52) U.S. Cl. 435/6; 702/20

(57) ABSTRACT

Provided are a method and a system for allergy analysis by using RNA derived from leukocytes extracted from peripheral blood as such, correcting the observed value, and comparing the balance of helper T (Th) cells (Th1/Th2) with the number of Th1 cells/the number of Th2 cells in a nucleic acid sample or the Th1/Th2 data of a patient and of a normal volunteer. Also provided are a method and a system for allergy analysis using a highly reproducible and reliable array having a minimal number of DNA fragments (oligonucleotides) thereon, which is realized by identifying essential gene groups for the Th1/Th2 assay. According to the present invention, it is possible to analyze the existence of a specific cell in a sample containing several types of cells without isolating the cell of interest.

### TECHNICAL FIELD

The present invention relates to a method and a system, for analyzing the existence of a specific cell in a sample containing several types of cells through gene expression by using an oligonucleotide array. More particularly, the present invention relates to a method and a system for allergy analysis for simple evaluation of allergy levels using an oligonucleotide array.

### BACKGROUND ART

Recently, increases in patients suffered from life-style related diseases or atopic or allergic diseases have become one factor for increased medical expenditures for people. Up to the present, administration of topical steroidal drugs has been the therapeutic method for allergic diseases, although this cannot inhibit the symptoms completely. On the other hand, desensitizing therapy has recently drawn attention. This method is carried out by administering an antigen extract (e.g., mite antigen) sublingually or the like. Desensitizing therapy is a therapeutic method for attenuating allergic reactions by repeatedly administering antigens to patients with specific allergens, in which mite, cedar pollen, or the like is used as the antigen. Compared to other types of drug therapy, side effects caused by desensitizing therapy is smaller, and if it works successfully, significant therapeutic effects can be attained. Thus, it is very beneficial for allergy patients. However, the success or failure of the therapy depends on the optimization of the antigen liquid concentration. The higher the antigen liquid concentration, the better the therapeutic effect. Side effects, however, should not be induced. At present, there is no method for determining the optimal antigen liquid concentration which varies for each individual. Further, there is no diagnostic index for determining whether the therapy should be continued or switched to other therapy when therapeutic effects are not apparent.

Allergy, however, is a serious symptom with which many patients are afflicted. It would be beneficial if allergy can be easily treated or diagnosed by general practitioners and the like, not to mention specialized medical institutions. From such a point of view, the development of diagnostic instruments, which are targeted to assess for appropriate levels of therapeutic measures for allergies and their therapeutic effects has been awaited.

It is considered that the benefits of the desensitizing therapy mentioned above are affected because the balance of helper T cells is changed and the migration of inflammatory cytokines is inhibited. Helper T cells can be classified into cell populations referred to as Th1 and Th2 depending on the types of cytokines generated therefrom, and they regulate different immune responses. Th1 cells are mainly involved with phylaxis through the cell-mediated immune responses against intracellular parasitic microorganisms, and Th2 cells are mainly involved with phylaxis through the humoral immune responses against extracellular parasitic microorganisms. These two types of cell populations maintain the equilibrium with each other through the cytokines produced by the other, thereby regulating each other to prevent excessive reactions from taking place. Many diseases such as allergic diseases developing due to the action of the Th1 or Th2 are considered to develop from imbalance in Th cells.

Up to the present, genes that are expressed differently in Th1 and in Th2 have been determined by DNA chips or Serial Analysis of Gene Expression (SAGE), and reported. Rogge et al. reported 215 genes that are expressed differently in Th1 and in Th2 using DNA chips having 6,000 genes placed thereon (Rogge, L. et al., Transcript imaging of the development of human T helper cells using oligonucleotide arrays, Nature Genetics 25, 96-101, 2000). Also, Nagai et al. (Nagai, S et al., Comprehensive gene expression profile of human activated Th1- and Th2-polarized cells, International Immunology 13, 367-376, 2001) analyzed 20,000 or more transcripts in Th1 cells and in Th2 cells respectively by SAGE, and reported 66 genes with higher levels of expression (critical rate of 5% or lower) in Th1 cells and 14 genes with higher levels of expression (critical rate of 5% or lower) in Th2 cells. Thus, from both reports, it is recognized that a wide variety of genes, which encode proteins such as cytokine, growth factor, receptor, transcription factor, cell adhesion-associated protein, cell migration-associated protein, ion channel, transporter, apoptosis-associated protein, signal transduction-associated protein, or metabolic pathway-associated protein, are expressed differently in Th1 cells and in Th2 cells. It should be noted that except for a few, the genes mentioned in these two reports are substantially different from each other.

In principle, a Th1/Th2 balance can be assayed by observing the distribution of the expression levels of the genes reported above. Such analyses, however, have never been carried out in the field of medical care. For one reason, the above-mentioned reports utilize the Th1 and Th2 cells previously isolated from blood by a given method. In actual medical practice, the amount of blood that can be obtained from a subject in one sampling is 5 to 10 cc. The amount of total RNA that can be extracted from this 5 to 10 cc of blood is not more than 10 μg with some inter-individual variations. In humans, since not more than 5% of the amount of total RNA is messenger RNA, the messenger RNA level that can be extracted from 5 to 10 cc of blood is only 0.5 μg. In addition, blood contains lymphocytes such as B cells and NK cells, neutrophils, eosinophils, basophils, and granulocytes such as monocytes/macrophages, as well as T cells. Accordingly, the messenger RNA level derived from Th1, Th2 cells that can be extracted from 5 to 10 cc of blood is 0.05 μg or lower, which is significantly smaller than the messenger RNA level commonly used in DNA chips, i.e., 26.0 μg. Specifically, it is technically very difficult to conduct routine examinations by extracting only Th1 and Th2 cells from the subject and further extracting RNA therefrom. Also, isolation of Th1 and Th2 cells from blood requires many processes, which in turn increases a cost in one diagnosis. Accordingly, it is not preferable from the viewpoint of medical economics. The recovery in each process cannot be always 100%, and thus, the amount of RNA that can be used in diagnosis decreases as the number of pretreatment processes increases.

In order to overcome the difficulties mentioned above, the Th1 cells:Th2 cells ratio is preferably determined by using RNA obtained from whole blood instead of RNA obtained from helper T cells only.

### SUMMARY OF THE INVENTION

An object of the present invention is to provide a method for analyzing the existence of a specific cell in a sample containing several types of cells without isolating the cell of interest. In one preferred embodiment, the present invention provides a method and a system for allergy analysis using an oligonucleotide array with the propriety of therapeutic measures and the level of their therapeutic effects can be assessed in a simple manner at low cost with high reliability. In a conventional analytical method by isolating T cells, T cell isolation and, in some cases, culture should be conducted. This increases the cost and time for analysis. Further the distribution of cellular gene expression may be affected by processes such as T cell isolation and culture. Thus, it is preferable not to carry out T cell isolation insofar as conditions permit. Accordingly, the present invention provides a method and a system for allergy analysis by using RNA derived from leukocytes extracted from peripheral blood as such, correcting, assessing the balance of the helper T (Th) cells (the Th1 cells:Th2 cells ratio (Th1/Th )), and comparing the value with the number of Th1 cells/the number of Th2 cells in a nucleic acid sample or Th1/Th2 data of a patient and of a normal volunteer. It is another object of the present invention to provide a method and a system for allergy analysis using a highly reproducible and reliable array having a minimal number of DNA fragments (oligonucleotides) thereon, which is realized by identifying gene groups that are essential for the Th1/Th2 assay.

We have conducted concentrated studies in order to attain the object of evaluating allergy levels using an oligonucleotide array. As a result, we found that, when assaying Th1 cells: Th2 cells ratio, the expression of genes in leukocyte: for example, (1) genes that are expressed specifically in Th cells; (2) genes that are expressed specifically in cells other than T cells, such as B cells and monocytes; (3) genes that are expressed equally in Th cells and in other cells; and (4) genes that are expressed differently in Th1 cells and in Th2 cells should be assayed preferably on the same array. In particular, when RNA obtained from whole blood is used, a preferred method is a method for determining the Th1 cells:Th2 cells ratio based on the genes which satisfy conditions (1) and (4) above, i.e., genes that are expressed specifically in Th cells and expressed differently in Th1 cells and in Th2 cells, and comprising correction of the expression level of (4), i.e., the expression level of genes that are expressed differently in Th1 cells and in Th2 cells using the expression levels of genes of (1) and (2), i.e., the expression level of genes that are expressed specifically in Th cells and genes that are not expressed in T cells and the expression level of genes of (3) that are expressed equally in Th cells and in other cells.

More specifically, the present invention provides a method for allergy analysis by observing the balance of the Th1 cells:Th2 cells ratio (Th1/Th2), comprising the steps of:

labeling a nucleic acid sample derived from human peripheral blood with fluorescence;

performing hybridization of the nucleic acid sample labelled with fluorescence with a plurality of probes, which contain genes that are expressed specifically in Th cells, genes that are expressed specifically in Th1 cells, and genes that are expressed specifically in Th2 cells;

detecting the presence or absence of hybridization by the fluorescence as the expression level;

assaying expression levels of one or more genes that are expressed specifically in Th1 cells and in Th2 cells respectively based on detected fluorescence intensity; and

comparing (the number of Th1 cells)/(the number of Th2 cells) in the nucleic acid sample or the Th1/Th2 data of a patient and of a normal volunteer with (Th1/Th2)=(the expression levels of one or more genes that are expressed specifically in Th1 cells)/(the expression levels of one or more genes that are expressed specifically in Th2 cells), thereby analyzing allergy.

When complementary DNA (cDNA) is synthesized by reverse transcription using, as a template, RNA obtained from leukocytes or T cells derived from human peripheral blood, cDNA is labelled by incorporating a labelling substance at the time of binding or extension, and the labelled cDNA can be used as a nucleic acid sample.

In this specification, “genes that are expressed specifically in cell A” refers to genes, that have expression levels that are statistically significant in cell A but below the lower limit of the measurable range or are statistically insignificant in cells other than cell A, which are contained in the specimen, according to the observation using, for example, a DNA chip or assay methods such as RT-PCR.

In the present invention, the number of genes that are expressed specifically in Th1 and in Th2, the number of genes that are expressed specifically in cells other than Th cells, and the number of genes that are expressed differently in Th1 and in Th2, are respectively 1 or more and preferably in the range of 50 to 200, for accurate analysis. The use of a larger number of genes enables more accurate analysis. If the number is too large, however, workloads in experiments or correction increase. Thus, the number of genes in the above-stated range is sufficient to obtain reliable values. Cells other than T cells include at least one cell selected from neutrophil, eosinophil, basophil, monocyte, macrophage, B cell, and NK cell.

In this specification, the term “expression level” refers to cellular levels of messenger RNA or cellular levels of protein. A sum, average, or median of the expression levels of all genes is used as the expression level of a plurality of genes. When the expression is assayed based on the fluorescence intensity, the median of the fluorescence intensity ranking among the plurality of probes can be employed.

According to the method of the present invention, when genes corresponding to s plurality of probes also contain genes that are expressed specifically in cells other than Th cells and the nucleic acid sample is also expressed in cells other than Th cells, the expression levels of one or more genes that are expressed specifically in Th1 cells and in Th2 cells can be respectively (fluorescence intensity)×(α/(α+1)) (α: the expression levels of one or more genes that are expressed specifically in Th cells/the expression levels of one or more genes that are expressed specifically in cells other than Th cells).

Further, the expression level for one or more genes that are expressed specifically in Th1 cells and in Th2 cells can be a sum or an average of the fluorescence intensity values of respective genes and the product with β (a normalizing function to realize Th1/Th2=1 when Th1:Th2 is 1:1) can be Th1/Th2.

Alternatively, the expression level for one or more genes that are expressed specifically in Th1 cells and in Th2 cells can be a median of the fluorescence intensity ranking among the fluorescence intensity values of the plurality of probes, respectively, and the product with β can be Th1/Th2.

The method according to the present invention can be used for assessing a disease status and/or disease level by, for example, assaying Th1/Th2 in the sample. The “specific state” refers to a control condition such as a normal state when, for example, a disease state and/or level of allergy and the like is to be analyzed. If Th1/Th2 is 1 in a normal state, statistical significance based on deviations from 1 is evaluated. Examples of statistical techniques that are used in the present invention include Bayesian estimation and Neyman-Pearson's estimation, without particular limitations.

In this specification, the term “expressed equally” refers to the level of gene expression with ±10%. Specifically, when the difference in the expression levels of a certain gene in two or more cells is within ±10%, the gene is referred to be “expressed equally” in the two or more cells.

In the method of the present invention, the number of genes used that are expressed equally in all of a pluraliy types of cells is 1 or more, and preferably 10 to 50.

According to the method of the present invention, changes with time in the ratio of the number of one or several types of cells to the number of entire cells can be analyzed, and whether or not the number of a specific type of cell increased or decreased in a sample comprising a plurality of types of cells can be analyzed without isolating the cell of interest.

Analysis of the Th1/Th2 value in a sample by the above method without the separation of cells from one another enables rapid assessment of whether, with respect to normal values, Th is imbalanced or not or the degree of imbalance thereof. Thus, information useful for therapy can be provided. Further, previous data accumulation for a patient and for a normal volunteer can express the correlation between the Th1/Th2 value and the disease rate as a threshold model. Therapeutic measures for whether or not the subject should be treated can be also determined based on the Th1/Th2 value obtained in each examination. Furthermore, the diagnostic accuracy can be enhanced by using an assessment method based on Bayesian Statistics or determination.

The present invention further provides a system for allergy analysis by observing the balance of the Th1 cells:Th2 cells ratio (Th1/Th2) comprising:

an oligonucleotide array having a plurality of probes, which correlate a plurality of genes comprising genes that are expressed specifically in Th cells, genes that are expressed specifically in Th1 cells, and genes that are expressed specifically in Th2 cells, immobilized thereon;

a detection means for detecting, as the expression level, the fluorescence of a nucleic acid sample hybridized with the probes;

a data storage means for storing data on the number of Th1 cells and the number of Th2 cells in the nucleic acid sample or Th1/Th2 of a patient and of a normal volunteer;

a data storage means for storing data on the expression levels of one or more genes that are expressed specifically in Th1 cells and in Th2 cells based on the detected fluorescence intensities; and

a computer for comparing (the number of Th1 cells)/(the number of Th2 cells) in the nucleic acid sample or the Th1/Th2 data of a patient and of a normal volunteer with (Th1/Th2)=(the expression level for one or more genes that are expressed in Th1 cells)/(the expression level for one or more genes that are expressed in Th2 cells).

This specification includes part or all of the contents as disclosed in the specification and/or drawings of Japanese Patent Application No. 2002-204785, which is a priority document of the present application.

### BRIEF DESCRIPTION OF DRAWINGS

FIG. 1 shows an example of positioning probe DNA on a substrate.

FIG. 2 shows a general structure of a DNA chip.

FIG. 3 shows an example in which a sample containing only Th1 and Th2 cells is assayed to evaluate Th1/Th2.

FIG. 4 shows an example in which a sample containing monocytes in addition to Th1 and Th2 cells are assayed to evaluate Th1/Th2.

FIG. 5 schematically shows the DNA chip production.

FIG. 6 shows a flow chart for the DNA chip production.

FIG. 7 schematically shows the DNA chip data analysis.

FIG. 8 shows a flow chart for the DNA chip data analysis.

FIG. 9 shows a flow chart indicating a method for selecting a suitable formula from among formulae 4-1 to 4-6.

### DESCRIPTION OF REFERENCE NUMERALS

1. substrate

2. probe DNA immobilized region

11. immobilized regions of DNA fragments (probe DNA) having gene sequences of genes that are expressed differently in Th1 cells and in Th2 cells or complementary sequence strands thereof

12. probe DNA of genes that are expressed specifically in Th cells

13. immobilized regions of probe DNA of genes that are not expressed very much in Th cells but are expressed specifically in cells other than T cells, such as B cells and monocytes

14. immobilized region of probe DNA of genes that are expressed equally in Th cells and in other cells

111. immobilized region of probe DNA of genes that are expressed differently in Th1 cells and in Th2 cells and the expression level in Th1 cells is higher than that in Th2 cells

112. immobilized region of probe DNA of genes that are expressed differently in Th1 cells and in Th2 cells and the expression level in Th2 cells is higher than that in Th1 cells

21. fluorescence detector

22. DNA probe

23. fluorescence-labelled gene

24. support

25. public database

26. in-house database

27. computer for designing probe sequence

28. probe sequence

29. nucleic acid synthesizer

30. oligonucleotide probe set

31. spotter

32. computer for controlling spotter

33. chip (production in progress)

34. chip (produced)

35. fluorescence-labelled sample

36. chip

37. fluorescence detector

38. computer for controlling fluorescence detector

39. experimental data

40. computer for analyzing experimental data

### PREFERRED EMBODIMENTS OF THE INVENTION

The present invention is hereafter described in more detail.

In this specification, an “internal control gene for proofreading” refers to, for example, a house keeping gene. The house keeping gene encodes, for example, a structural protein that is necessary for cellular survival or an enzyme in the energy metabolic system, and it is considered to be expressible in any cell with various types of differentiation. Examples thereof include β-actin, GAPDH, HPRT, alpha-tublin, a transferrin receptor, and ubiquitin. Since these are already contained in the sample from the subject, such as leukocytes, they can be internal controls at the time of proofreading. These internal controls are already contained in the sample and become controls at the time of proofreading.

In this specification, an “external control gene for proofreading” refers to, for example, when a human is to be assayed, a gene sequence from plants, microorganisms, insects, or the like that is absent in humans. Examples thereof include Arabidopsis thaliana gene, plasmid DNA, bacteriophage DNA, and Firefly luciferase gene. Since it is not contained in the sample from the subject, such as leukocytes, it can be an external control at the time of proofreading when a known concentration of this external control gene is externally added at the time of assay. The external control is not previously present in the sample, and thus, it becomes a control at the time of proofreading by being externally and separately added.

T cells are described. T cells are also referred to as T-lymphocytes, they are a subpopulation that accounts for 60 to 80% of the peripheral blood lymphocytes, and they are derived from bone marrow stem cells. Most thereof mature in the thymes gland. Many mature T cells become helper T (Th) cells or killer T cells. The helper T cells produce various cytokines by antigen stimulation, and act on B cells, T cells, macrophages, and the like to induce acceleration and potentiation of immune response. The killer T cells are capable of recognizing and attacking transplants, virus infected cells, and tumor cells. Scientifically, it is known that the STAT4, IL12RB2, TBX21, IFNG, ERM, IL18, JAK2, TYK2, IL18R, IL2, HLX, and IRF1 genes are essential for Th1 cell differentiation. SCYC1, SPP1, MIP1A, MIP1B, PRF1, CTNNB1, CXCR3, CCR5, RANTES, and NFKB are genes that are not always necessary in Th1 cell differentiation but scientifically expected to be involved therewith, and to be expressed differently in Th1 cells and in Th2 cells. Also known is the SOCS1 gene that is considered to inhibit differentiation to Th2 cell upon Th1 cell differentiation.

Similarly, it is scientifically known that the STAT6, GATA3, MAF, IL13IL5, IL4, IL6, IL10, NFAT, and NIP45 genes are essential in Th2 cell differentiation. FUS, ILF2, E3-EPF, PSG, CRTH2, and CCR4 are genes that are not always necessary in Th2 cell differentiation but scientifically expected to be involved therewith, and to be expressed differently in Th1 cells and in Th2 cells. Also known is the SOCS3 gene that is considered to inhibit differentiation to Th1 cell upon Th2 cell differentiation.

It is scientifically considered that the IL12RB1, IL4RA, IL3, and GM-CSF genes are expressed substantially equally in Th1 cells and in Th2 cells.

The balance maintained by the cytokines generated by Th1 cells and Th2 cells can be expressed at a genetic level as follows. If several groups of Th1/Th2 associated genes are switched, the level of the Th1/Th2 associated protein are either increased or decreased, and the balance of these protein activities, as a whole, is considered to regulate biofunctions. Specifically, abnormal switching of groups of Th1/Th2 associated genes results in imbalance of protein activities. This manifests as a regulatory abnormality of biofunctions such as Th1/Th2. Switching of genes is regulated by, for example, an increase or decrease in the gene expression level. The gene expression level can be assayed by employing a messenger RNA level or a protein level as an index. In currently available techniques, the assay can be carried out much more easily by employing, as an index, the messenger RNA level instead of the protein level. For simple evaluation of allergy, an increase/decrease in the expressed messenger RNA level of a plurality of Th1/Th2 associated genes may be simultaneously observed. To this end, the DNA array (this may be referred to as an “oligonucleotide array”) is most suitable when the number of associated genes is more than 100 while the DNA array or the Reverse Transcription-Polymerase Chain Reaction (RT-PCR) is suitable when the number of associated genes is not more than 100. In the present invention, the “DNA array” and the “oligonucleotide array” include those prepared by synthesizing oligonucleotides on a support and those prepared by spotting amplified gene fragments (oligonucleotides) on a support. It should be noted that the processes for producing those are not specified by their names.

The DNA array (oligonucleotide array) comprises a plurality of DNA fragments (oligonucleotides) immobilized on a substrate. Each oligonucleotide independently corresponds to a different gene. When assaying, messenger RNA derived from an assay sample is employed as a template for reverse transcription, thereby synthesizing a complementary DNA (cDNA) fragment. When only a small amount of sample is available, it may be amplified by PCR or the like. At the time of the reverse transcription or PCR, a suitable labelling substance such as a fluorescent substance is incorporated upon binding or extension, thereby labelling the cDNA fragment (hereinafter, cDNA with a label is referred to as the “labelled cDNA”).

The oligonucleotide that is immobilized on a substrate is bound complementarily with the labelled cDNA fragment. Each oligonucleotide is immobilized on the substrate at different coordinates. If the types of oligonucleotides immobilized at given coordinates can be previously identified, a decrease/increase in messenger RNA for a plurality of individual genes can be simultaneously assayed.

RT-PCR is a method for assaying the expression of the subject genes by employing genes with small intercellular variations in the expression level (e.g., house keeping genes) as a standard. Since the messenger RNA derived from an assay sample can be amplified by RT-PCR, a smaller amount of sample than the DNA array method suffices for the assay.

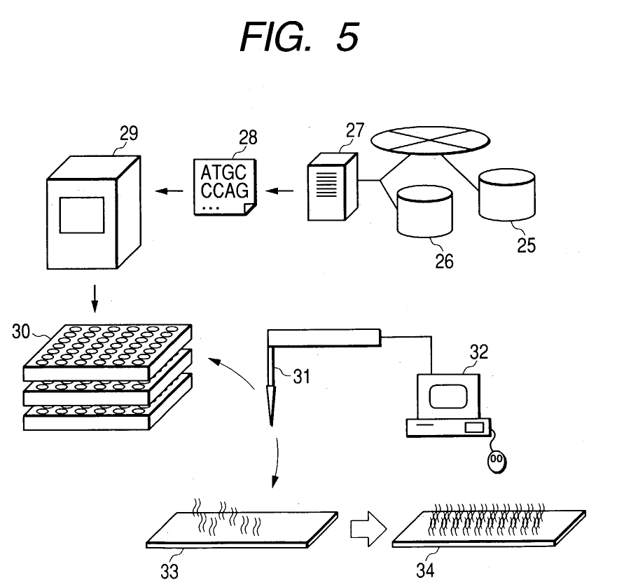

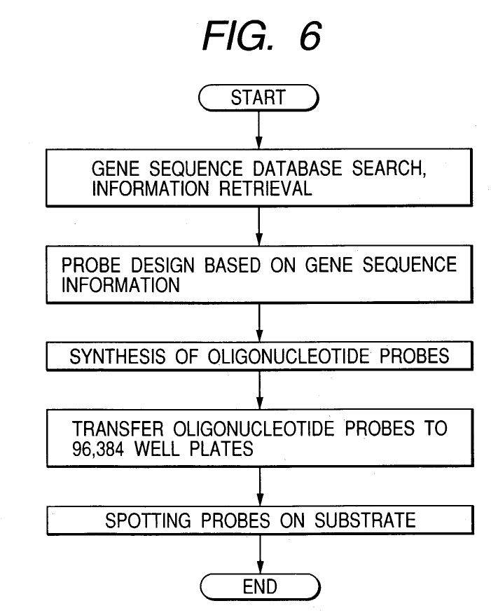

FIG. 5 shows a general scheme for the production of DNA chips, and the flow chart therefor is shown in FIG. 6. Gene information (such as sequence information and functional information) are first obtained from public database 25 accessible from a network such as the Internet or in-house database 26. Based on these pieces of gene information, probe sequences for specific genes are designed, and these sequences are designed by computer 27 for designing probe sequences. The computed nucleotide sequence 28 is input into nucleic acid synthesizer 29 to synthesize oligonucleotide probes. The synthesized probe set 30 is spotted on a support of DNA chip (production in progress) 33 using spotter 31, thereby preparing DNA chip 34.

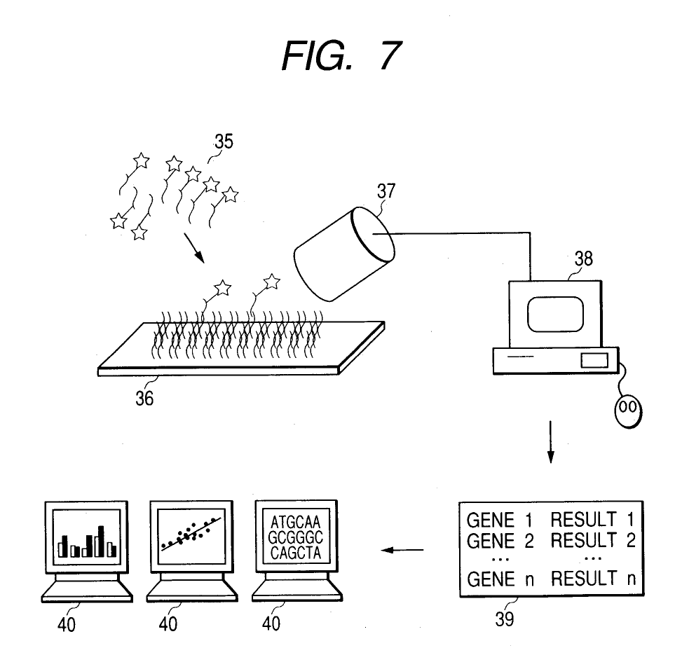

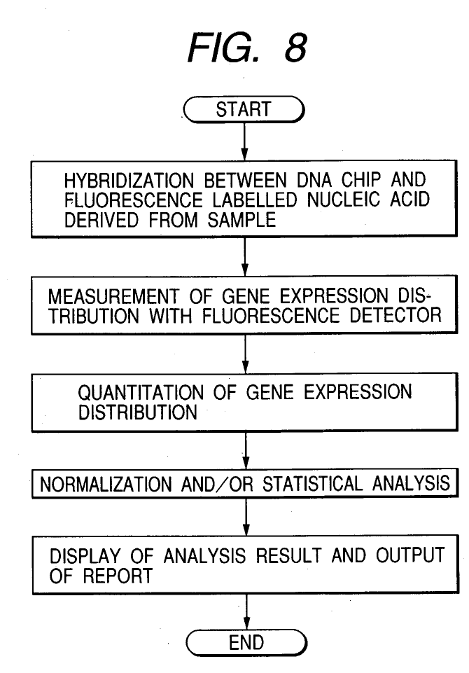

FIG. 7 schematically shows the analysis of DNA chip data, and the flow chart therefor is shown in FIG. 8. A nucleic acid sample obtained from the subject is first labelled with a fluorescent label to prepare fluorescence-labelled sample 35. The fluorescence-labelled sample 35 is then hybridized onto chip (oligonucleotide array) 36 comprising a plurality of probes (oligonucleotides), immobilized thereon, which can be hybridized with a nucleic acid sample in a sequence-specific manner. After hybridization, the chip 36 is screened using fluorescence detector (detection means) 37 to obtain fluorescence levels (fluorescence intensity) corresponding to each probe. The obtained fluorescence level is stored in computer 38 for regulating fluorescence detector (data storing means) as experimental data 39 (data on distribution of gene expression). Subsequently, the experimental data 39 is transferred to computer 40 for analyzing experimental data to perform correction or statistical analysis by the formulae by the method disclosed in this specification or the like. The results of the analysis are provided to the analyzer in the form of a screen output or report output.

When a nucleic acid sample derived from a subject with allergy or with suspected allergy is used as a sample, the allergy status and/or level can be analyzed by the above method or system.

The present invention provides a method and a system of analysis using an oligonucleotide array comprising a plurality of oligonucleotides with different nucleotide sequences immobilized onto a support at different locations, wherein the oligonucleotides have a nucleotide sequence comprising at least 20 consecutive nucleotides of the genes according to (1) to (4) below or complementary sequence strands of the genes described below.

More specifically, groups (1) to (4) refer to:

(1) an oligonucleotide derived from genes that are expressed specifically in cell 1 and in cell 2 or an oligonucleotide that is complementary to the oligonucleotide mentioned just above;

(2) an oligonucleotide derived from genes that are expressed specifically in cell k (k=3, 4, . . . , n) or an oligonucleotide that is complementary to the oligonucleotide mentioned just above;

(3) an oligonucleotide derived from genes that are expressed equally in all the n types of cells or an oligonucleotide that is complementary to the oligonucleotide mentioned just above; and

(4) an oligonucleotide derived from genes that are expressed differently in cell 1 and in cell 2 or an oligonucleotide that is complementary to the oligonucleotide mentioned just above.

The number of oligonucleotides to be immobilized which belong to each group in the oligonucleotide array is not particularly limited. Preferably, the numbers thereof for groups (1), (2), and (3) are respectively in the range of 10 to 50, and in the range of 50 to 200 for group (4).

When immobilizing oligonucleotides on a substrate, they are preferably positioned separately depending on their groups. Thus, the assay results obtained by the oligonucleotide array can be instantaneously and visually understood and assessed by the examiner.

The present invention also provides to a method and a system of analysis using an oligonucleotide array comprising oligonucleotides (equivalent to (4)) immobilized thereon, each having a nucleotide sequence comprising at least 20 consecutive nucleotides in genes that are expected to be expressed differently in Th1 cells and in Th2 cells or a complementary sequence thereof. Genes that are expressed differently in Th1 cells and in Th2 cells include a wide variety of genes, which encode cytokine, chemokine, growth factor, transcription factor, apoptosis-associated protein, proteolysis-associated protein, signal transduction-associated protein, enzyme, ion channel, transporter, metabolism-associated protein, cell adhesion-associated protein, cell migration-associated protein, and allergic protein. Specifically, in terms of Symbol designations of the Unigene, these genes include, for example, ADCY7, AGXT, AKAP1, ALDH3B1, ANXA3, ARCN1, AREG, ARRB2, ATP1A1, ATP6D, ATP6F, BAK1, BIRC3, CACNB3, CASP8, CCR4, CCR5, CD2, CD38, CD3D, CD6, CD69, CD97, CDH3, CEBPD, CNK, COPS3, COX5B, CRI1, CSF2, CST3, CTNNB1, CXCR3, DAF, DNTT, DPP4, DTR, E2-EPF, E2F4, EBI2, EDG1, EGR2, ERM.ETS1, FACL1, FGD1, FGFR1, FUS, FLJ14639, FLJ20746, FOS, FOXG1A, FUS, GATA1, GATA3, GBP1, GBP2, GC20, GCH1, GNLY, GOT1, GPR6, GPR9, GZMB, GZMK, HIF1A, HLX, HNRPA1, HOXA1, HSP105B, HSPA1A, HSPCA, HSXIAPAF1, HT013, ICAM2, ICSBP1, IFI35, IFNG, IL2, IL4, IL5, IL6, IL10, IL10RA, IL12RB2, IL13, +IL18R1, IL2, IL3, IL9, IL13, IL18, IL18R, ILF2, IRF1, IRF7, ITGB7, ITK, JAK2, JUN, KCNB2, KCNK3, KIAA0239, KLK6, KSR, LDHB, LEP, LIFR, LOC51042, LOC64116, LRP8, LTA, LTB, MAF, MAP2K2, MAP3K5, MIG, MIP, MKNK1, MRF-1, MSN, MT1H, MYB, NDUFB5, NFATC1, NFKB, NKG7, NME4, NR4A2, NT5, OSM, P2RX5, PDCD5, PDE4B, PEF, PEMT, PheHB, IM1, PLAUR, PP, PPIF, PPP1R2, PPP2R5D, PRDX1, PRF1, PRKCB1, PRKCH, PSCDBP, PSMB4, PSMC4, PSMC6, PTB, PTGER4, PTPRA, PTPRZ1, RAB32, RDBP, RORA, RPS24, SCYA15, SCYA17, SCYA3, SCYA4, SCYA5, SCYC1, SDH, SELL, SERPINB1, SET, SF3A3, SFRS10, SFRS5, SLA, SOCS1, SOCS3, SPP1, SRM300, STAT1, STAT4, STAT6, STIP1, TAF2S, TANK, TAP1, TBX21, TBXA2R, TIEG, TIMP1, TNFRSF1A, TNFRSF1B, TNFSF10, TNFSF5, TYK2, UROD, USF2, USP12, USP7, XBP1, YME1L1, and YR-29. Examples of these genes are shown in Tables 1 to 6 together with their accession numbers in the GenBank.

TABLE 1

| Unigene Symbol | RefSec(GenBank) No. |
|---|---|
| ADCY7 | NM_001114 |
| AGXT | NM_000030 |
| AKAP1 | NM_003488 |
| ALDH3B1 | NM_000694 |
| ANXA3 | NM_005139 |
| ARCN1 | NM_001655 |
| AREG | NM_001657 |
| ARRB2 | NM_004313 |
| ATP1A1 | NM_000701 |
| ATP6D | L05087 |
| ATP6F | NM_004047 |
| BAK1 | NM_001188 |
| BIRC3 | NM_001165 |
| CACNB3 | NM_000725 |
| CASP8 | NM_001228 |
| CD2 | NM_001767 |
| CD38 | NM_001775 |
| CD3D | NM_000732 |
| CD6 | NM_006725 |
| CD69 | NM_001781 |
| CD97 | NM_001784 |
| CDH3 | NM_001793 |
| CEBPD | NM_005195 |
| CNK | NM_004073 |
| COPS3 | NM_003653 |
| COX5B | NM_001862 |
| CRI1 | NM_014335 |
| CSF2 | NM_000758 |
| CST3 | NM_000099 |
| CTNNB1 | NM_001904 |

TABLE 2

| Unigene Symbol | RefSec(GenBank) No. |
|---|---|
| DAF | NM_000574 |
| DNTT | NM_004088 |
| DPP4 | NM_001935 |
| DTR | NM_001945 |
| E2-EPF | NM_014501 |
| E2F4 | NM_001950 |
| EBI2 | NM_004951 |
| EDG1 | NM_001400 |
| EGR2 | NM_000399 |
| ETS1 | NM_005238 |
| FACL1 | NM_001995 |
| FGD1 | NM_004463 |
| FGFR1 | NM_000604 |
| FLJ20746 | NM_019025 |
| FOS | NM_005252 |
| FOXG1A | NM_004471 |
| FUS | NM_004960 |
| GATA1 | NM_002049 |
| GATA3 | NM_002051 |
| GBP1 | NM_002053 |
| GBP2 | NM_004120 |
| GC20 | NM_005875 |
| GCH1 | NM_000161 |
| GNLY | NM_006433 |
| GOT1 | NM_002079 |
| GPR6 | NM_005284 |
| GPR9 | NM_001504 |
| GZMB | NM_004131 |
| GZMK | NM_002104 |
| HIF1A | NM_001530 |

TABLE 3

| Unigene Symbol | RefSec(GenBank) No. |
|---|---|
| HNRPA1 | NM_002136 |
| HOXA1 | NM_005522 |
| HSP105B | NM_006644 |
| HSPA1A | NM_005345 |
| HSPCA | NM_005348 |
| HSXIAPAF1 | NM_017523 |
| HT013 | NM_018474 |
| ICAM2 | NM_000873 |
| ICSBP1 | NM_002163 |
| IFI35 | U72882 |
| IFNG | NM_000619 |
| IL10RA | NM_001558 |
| IL12RB2 | NM_001559 |
| IL13 | NM_002188 |
| IL18R1 | NM_003855 |
| IL2 | NM_000586 |
| IL3 | NM_000588 |
| IL9 | NM_000590 |
| ILF2 | NM_004515 |
| IRF1 | NM_002198 |
| IRF7 | NM_001572 |
| ITGB7 | NM_000889 |
| ITK | NM_005546 |
| JUN | NM_002228 |
| KCNB2 | NM_004770 |
| KCNK3 | NM_002246 |
| KIAA0239 | NM_015288 |
| KLK6 | NM_002774 |
| KSR | U43586 |
| LDHB | NM_002300 |

TABLE 4

| Unigene Symbol | RefSec(GenBank) No. |
|---|---|
| LEP | NM_000230 |
| LIFR | NM_002310 |
| LOC51042 | NM_015871 |
| LOC64116 | NM_022154 |
| LRP8 | NM_017522 |
| LTA | NM_000595 |
| LTB | NM_002341 |
| MAP2K2 | NM_030662 |
| MAP3K5 | NM_005923 |
| MIG | NM_002416 |
| MKNK1 | NM_003684 |
| MRF-1 | M62324 |
| MSN | NM_002444 |
| MT1H | NM_005951 |
| MYB | NM_005375 |
| NDUFB5 | NM_002492 |
| NFATC1 | NM_006162 |
| NKG7 | NM_005601 |
| NME4 | NM_005009 |
| NR4A2 | NM_006186 |
| NT5 | NM_002526 |
| OSM | NM_020530 |
| P2RX5 | NM_002561 |
| PDCD5 | NM_004708 |
| PDE4B | NM_002600 |
| PEF | NM_012392 |
| PEMT | NM_007169 |
| PheHB | NM_005687 |
| PIM1 | NM_002648 |
| PLAUR | NM_002659 |

TABLE 5

| Unigene Symbol | RefSec(GenBank) No. |
|---|---|
| PP | NM_021129 |
| PPIF | NM_005729 |
| PPP1R2 | NM_006241 |
| PPP2R5D | NM_006245 |
| PRDX1 | NM_002574 |
| PRF1 | NM_005041 |
| PRKCB1 | NM_002738 |
| PRKCH | NM_006255 |
| PSCDBP | NM_004288 |
| PSMB4 | NM_002796 |
| PSMC4 | NM_006503 |
| PSMC6 | NM_002806 |
| PTB | NM_002819 |
| PTGER4 | NM_000958 |
| PTPRA | NM_002836 |
| PTPRZ1 | NM_002851 |
| RAB32 | NM_006834 |
| RDBP | NM_002904 |
| RORA | NM_002943 |
| RPS24 | NM_001026 |
| SCYA15 | NM_032964 |
| SCYA17 | NM_002987 |
| SCYA3 | NM_002983 |
| SCYA4 | NM_002984 |
| SCYA5 | NM_002985 |
| SCYC1 | NM_002995 |
| SDH | NM_004168 |
| SELL | NM_000655 |
| SERPINB1 | NM_030666 |
| SET | NM_003011 |

TABLE 6

| Unigene Symbol | RefSec(GenBank) No. |
|---|---|
| SF3A3 | NM_006802 |
| SFRS10 | NM_004593 |
| SFRS5 | NM_006925 |
| SLA | NM_006748 |
| SPP1 | NM_000582 |
| SRM300 | NM_016333 |
| STAT1 | NM_007315 |
| STIP1 | NM_006819 |
| TAF2S | NM_006706 |
| TANK | NM_004180 |
| TAP1 | NM_000593 |
| TBXA2R | NM_001060 |
| TIEG | NM_005655 |
| TIMP1 | NM_003254 |
| TNFRSF1A | NM_001065 |
| TNFRSF1B | NM_001066 |
| TNFSF10 | NM_003810 |
| TNFSF5 | NM_000074 |
| UROD | NM_000374 |
| USF2 | NM_003367 |
| USP12 | AF022789 |
| USP7 | NM_003470 |
| XBP1 | NM_005080 |
| YME1L1 | NM_014263 |
| YR-29 | NM_014886 |

The present invention also provides a method and a system of analysis using an oligonucleotide array comprising oligonucleotides (equivalent to (1)) immobilized thereon, each having a nucleotide sequence comprising at least 20 consecutive nucleotides in genes that are expressed specifically in Th cells or a complementary sequence thereof. Such oligonucleotides include those derived from genes, for example, in terms of Symbol designations of the Unigene, CD2, CD4, CD28, CD3D, CD3E, CD3G, CD3Z, CD8A, CD62L, CTLA4, MIC2, SEMA4D, THY1, TCRA, and TCRB. Also, CD44 is known as a gene that is expressed in Th cells. Examples of these genes are shown in Table 7 together with their accession numbers in the GenBank.

TABLE 7

| Unigene Symbol | RefSec(GenBank) No. |
|---|---|
| CD2 | NM_001767 |
| CD28 | NM_006139 |
| CD3D | NM_000732 |
| CD3E | NM_000733 |
| CD3G | NM_000073 |
| CD3Z | NM_000734 |
| CD8A | NM_001768 |
| CTLA4 | NM_005214 |
| MIC2 | NM_002414 |
| SEMA4D | NM_006378 |
| THY1 | NM_006288 |
| TRA@ | M12959 |
| TRB@ | X00437 |

The present invention also provides a method and a system of analysis using an oligonucleotide array comprising oligonucleotides (equivalent to (2)) immobilized thereon, each having a nucleotide sequence comprising at least 20 consecutive nucleotides in genes that are not expressed very much in Th cells but are expressed in cells other than T cells, such as B cells or monocytes, or a complementary sequence thereof.

Such oligonucleotides include those derived from genes, for example, in terms of Symbol designations of the Unigene, ADAM8, ANPEP, BST1, CD14, CD19, CD22, CD33, CD34, CD36, CD68, CD72, CD74, CD79A, CD79B, CD81, CD86, CEACAM1, CEACAM3, CEACAM4, CEACAM5, CEACAM8, CSF1R, CSF2RA, CSF2RB, CSF3R, FCGR1A, FCGR3A, FCGR3B, FLT3, GP1BA, GP1BB, GP5, GP9, ICAM2, IGSF2, IL3RA, IL5RA, IL12, IL27, ITGA2, ITGAM, ITGAV, ITGB3, ITGB4, KIT, LAMP1, LAMP2, LRP1, MS4A2, MST1R, NCAM1, PECAM1, PLAUR, PVR, SELP, SEMA7A, and VCAM1. Examples of these genes are shown in Tables 8 and 9 together with their accession numbers in the GenBank.

TABLE 8

| Unigene Symbol | RefSec(GenBank) No. |
|---|---|
| ADAM8 | NM_001109 |
| ANPEP | NM_001150 |
| BST1 | NM_004334 |
| CD14 | NM_000591 |
| CD19 | NM_001770 |
| CD22 | NM_001771 |
| CD33 | NM_001772 |
| CD34 | NM_001773 |
| CD36 | NM_000072 |
| CD68 | NM_001251 |
| CD72 | NM_001782 |
| CD74 | NM_004355 |
| CD79A | NM_001783 |
| CD79B | NM_000626 |
| CD81 | NM_004356 |
| CD86 | NM_006889 |
| CEACAM1 | NM_001712 |
| CEACAM3 | NM_001815 |
| CEACAM4 | NM_001817 |
| CEACAM5 | NM_004363 |
| CEACAM8 | NM_001816 |
| CSF1R | NM_005211 |
| CSF2RA | NM_006140 |
| CSF2RB | NM_000395 |
| CSF3R | NM_000760 |
| FCGR1A | NM_000566 |
| FCGR3A | NM_000569 |
| FCGR3B | NM_000570 |
| FLT3 | NM_004119 |
| GP1BA | NM_000173 |

TABLE 9

| Unigene Symbol | RefSec(GenBank) No. |
|---|---|
| GP1BB | NM_000407 |
| GP5 | NM_004488 |
| GP9 | NM_000174 |
| ICAM2 | NM_000873 |
| IGSF2 | NM_004258 |
| IL3RA | NM_002183 |
| IL5RA | NM_000564 |
| ITGA2 | NM_002203 |
| ITGAM | NM_000632 |
| ITGAV | NM_002210 |
| ITGB3 | NM_000212 |
| ITGB4 | NM_000213 |
| KIT | NM_000222 |
| LAMP1 | NM_005561 |
| LAMP2 | NM_002294, NM_013995 |
| LRP1 | NM_002332 |
| MS4A2 | NM_021950 |
| MST1R | NM_002447 |
| NCAM1 | NM_000615 |
| PECAM1 | NM_000442 |
| PLAUR | NM_002659 |
| PVR | NM_006505 |
| SELP | NM_003005 |
| SEMA7A | NM_003612 |
| VCAM1 | NM_001078 |

The present invention also provides to a method and a system of analysis using an oligonucleotide array comprising oligonucleotides (equivalent to (3)) immobilized thereon, each having a nucleotide sequence comprising at least 20 consecutive nucleotides in genes that are expressed equally in Th cells and in other cells or a complementary sequence thereof. Such oligonucleotides include, for example, those derived from internal control genes already contained in the sample from the subject, such as leucocytes, or those derived from external control genes prepared by adding a predetermined amount of gene sequences from plants, microorganisms, insects, or the like that are absent in humans to the assay sample at the time of assay. Specific examples include those derived from genes, for example, in terms of Symbol designations of the Unigene, AP1B1, AP1G1, AP1S1, AP1S2, AP1M2, CAMK1, CAMK2A, CAMK2B, CAMK2D, CAMK2G, CD28, CD47, CR1, CREB1, CTLA4, IFNGR1, IL1R1, IL1R2, IL2RB, IL6ST, ITGA1, ITG26, ITGA3, ITGA4, ITGA5, ITGA6, LAT, MAPK13, MAPK2K4, MAPK2K7, NFKB1, NFKB2, RAF1, SLC326, TNFRSF1A, TNFRSF1B, and ZAP70.

In order to evaluate allergy levels, for example, the Th1 cells:Th2 cells ratio should be analyzed with high accuracy. Accordingly, it is obvious that the DNA fragment, which is supposed to be complementarily bound to 1 type of gene only should not be bound to other types of genes (cross hybridization). This becomes more complicated as the number of genes to be immobilized on one array increases. Thus, it is very difficult to completely avoid cross hybridization between genes on the DNA array for screening, which has 5,000 to several tens of thousands genes. As a result of analyses of sequence homology based on the BLAST algorithm, when the number of nucleotides in a DNA fragment to be used as a probe is not more than 1,000, it was found preferable to place 1,000 to 1,500 types or fewer DNA fragments on one array. Accordingly, if the DNA array is used to evaluate and diagnose allergy levels, it is preferable to prepare an array with a minimal number of only genes that are associated with the action mechanism of the allergy. It is not preferable to place genes unassociated with the allergy because it results in increased production costs for probes and increased prices for the oligonucleotide arrays. Because the number of oligonucleotide types to be used as probes for the array can be kept low, oligonucleotides of one type can be immobilized on several locations as probes. Thus, reliability can be enhanced by averaging the signal intensities at several locations.

More specifically, the use of a DNA array is most suitable for analyzing allergy levels, wherein the DNA array comprises, immobilized thereon, a minimal number of oligonucleotide probes respectively derived from:

(1) genes that are expressed specifically in Th cells;

(2) genes that are not expressed very much in Th cells but are expressed specifically in cells other than T cells, such as B cells and monocytes;

(3) genes that are expressed equally in Th cells and in other cells; and

(4) genes that are expressed differently in Th1 cells and in Th2 cells.

In order to position the oligonucleotides having sequences derived from the above genes or complementary sequences thereof on an array as probes, it is necessary to determine which portions in the gene sequences should be set as probes. In these cases, melting temperature (Tm) and the absence of cross hybridization should be taken into consideration. In order to hybridize each DNA fragment that are immobilized on the DNA array with each sample-derived DNA fragments with high accuracy (or under highly stringent conditions), the correlation between hybridization temperature (Th) and Tm of the immobilized DNA fragment is important. Specifically, the difference between melting temperature and hybridization temperature of the immobilized DNA fragment should not exceed 30° C. Since cross hybridization occurs due to high homology between DNA sequences, homology between sample-derived DNA fragment to be hybridized with specific immobilized DNA fragment and DNA fragments among sample-derived DNA fragments that are not inherently hybridized with the immobilized DNA fragments should be low enough in order to prevent cross hybridization. It is preferable that sequences which have mini-hairpin molecular structures, or portions, which are significantly homologous to a repeated sequence known as the Alu sequence in human gene, are not present. In addition to computing the homology among gene sequences immobilized on one array, homology between the DNA sequences of a given living species and gene sequences of a target living species registered in GenBank and the like should be computed. Based on the comparison between the sequences of the candidate DNA fragments immobilized on the DNA array and the DNA sequences of a given gene group which may be contained in an assay sample, it is preferable not to select significantly homologous DNA sequences as DNA fragments to be immobilized.

A DNA fragment to be immobilized as a probe can be synthesized by PCR using any commercially available cDNA library as a template. The synthesized product is adjusted to a predetermined concentration (0.1-1.0 μg/μl) and spotted on a poly-lysine or amino-silane coated slide glass by using a spotter. Thus, an oligonucleotide array can be prepared. Alternatively, a probe can be directly synthesized on a support by any known technique in the art.

Allergy levels can be assessed using the oligonucleotide array in the manner described below. At the outset, peripheral bloods are sampled from several individual volunteers who had no allergy symptom, and total RNA or messenger RNA are extracted from the leukocytes. For example, average messenger RNAs from normal volunteers are pooled together by mixing messenger RNAs of several volunteers. This pool of messenger RNAs is hereinafter referred to as “universal control” in this specification. Subsequently, peripheral blood is sampled from a subject, and messenger RNA is extracted from leukocytes. Reverse transcription for messenger RNA obtained from the subject's peripheral blood is carried out using oligo dT primer, and labelled cDNA is synthesized using Cy5-dCTP or the like. Regarding messenger RNA of the universal control, labelled cDNAs having different labels are synthesized using Cy3-dCTP or the like. The subject's cDNA (Cy5-labelled) and the universal control cDNA (Cy3-labelled) are mixed with each other and subjected to the same oligonucleotide array. Thereafter, they are allowed to hybridize with each other at a predetermined temperature for a predetermined length of time. Preferably, hybridization temperature is 45 to 70° C., and hybridization time is for 6 to 18 hours. After the hybridization, the fluorescence intensities of Cy5 and Cy3 at the sites where each gene had been spotted are compared by a fluorescence scanner, thereby determining the difference in the expression levels therebetween.

The genes of (1), (2), (3), and (4) can be assessed by RT-PCR or other methods such as Northern hybridization to determine the Th1 cells:Th2 cells ratio and the differences in the expression levels of cytokines therefrom. A method for computing the expression ratio of Th1/Th2 using (1) to (4) above is described below.

Several formulae, which are necessary for describing the correction method used herein, are first defined.

In this specification, Th1/Th2 is either:

Th1/Th2=the number of Th1 cells/the number of Th2 cells (formula 1)

or

Th1/Th2=the expression level for one or more genes expressed in Th1 cells/the expression level for one or more genes expressed in Th2 cells (formula 2).

The abundance ratio of Th cells to monocytes (hereinafter it may be referred to as “α”) can be defined by formula 3.

Abundance ratio of Th cells to monocytes (α)=the number of Th cells/the number of monocytes=(expression level for one or more genes that are expressed specifically in Th cells/expression level for one or more genes that are expressed equally in Th cells and in monocytes)/(expression level for one or more genes that are expressed specifically in monocytes/expression level for one or more genes that are expressed equally in Th cells and in monocytes) (formula 3)

Th1/Th2 can be determined as follows:

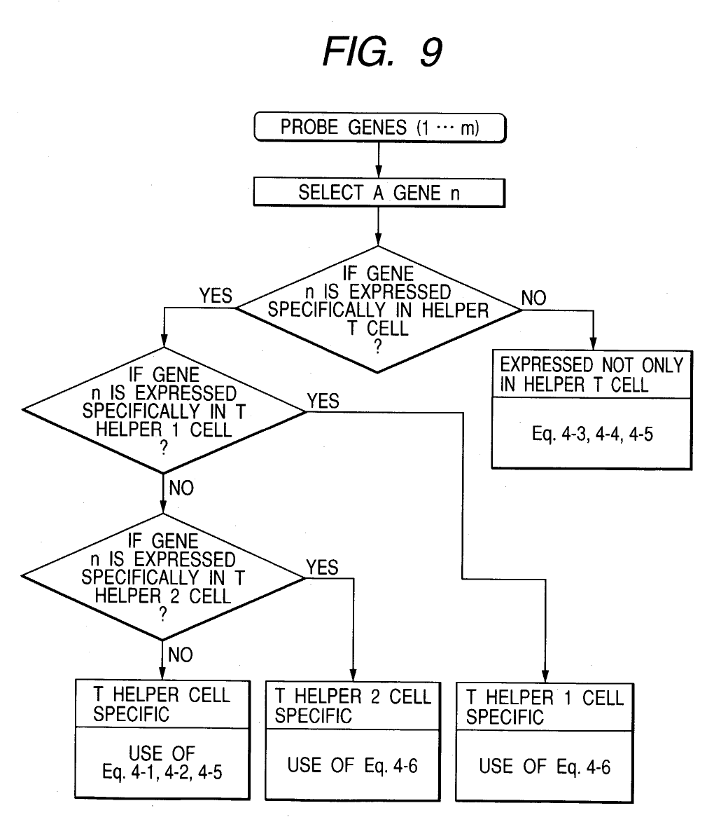

Specifically, there are presumed to be m number of probes on a chip substrate. A different gene corresponds to each probe. A formula is selected in accordance with a flow chart shown in FIG. 9 depending on whether or not the gene “n” to which a given probe corresponds (described as a “probe gene” in FIG. 9) is expressed only in Th cells (Th cell-specific), expressed in cells other than Th cells, expressed only in Th1 cells (Th1 cell-specific), or expressed only in Th2 cells (Th2 cell-specific).

As shown in FIG. 9, for example, the formula to be used varies depending on whether the gene “n” among the m number of probes is Th cell-specific or not. When it is also expressed in cells other than Th cells, formulae 4-3 to 4-5 are used.

When the gene “n” is Th cell-specific and is expressed specifically in either Th1 cells or Th2 cells, formula 4-6 is used.

When the gene “n” is Th cell-specific and is expressed in both Th1 and Th2 cells, formulae 4-1, 4-2, and 4-5 are used.

Specific formulae 4-1 to 4-6 are shown below.

Th1(n)=the expression level of gene “n” in Th1 cells (fluorescence intensity obtained from gene) (formula 4-1)

Th2(n)=the expression level of gene “n” in Th2 cells (fluorescence intensity obtained from gene) (formula 4-2)

Th1(n)=the expression level of gene “n” in Th1 cells (fluorescence intensity obtained from gene)×(α/(α+1)) (formula 4-3)

Th2(n)=the expression level of gene “n” in Th2 cells (fluorescence intensity obtained from gene)×(α/(α+1)) (formula 4-4)

If the total number of genes to be assayed is “k,” Th1/Th2 is determined by the following formula.

$$Th1 / Th2 = {\sum }_{n = 1}^{k} Th1 ( n ) / {\sum }_{n = 1}^{k} Th2 ( n ) \times β ( p , q , r ⋯ ) \qquad ( formula 4 \text{-} 5 )$$

β(p, q, r . . . ) is a function for normalizing Th1/Th2 to be equal to 1 when Th1:Th2 is 1:1, and it is determined by experiments. When β is experimentally determined, a group of previously adjusted samples is prepared to bring the Th1:Th2 to a known ratio, for example, 9:1, 8:2, 7:3, 6:4, 5:5, 4:6, 3:7, 2:8, or 1:9. Each sample adjusted to the known ratio is used to assay Th1/Th2. Subsequently, the previously adjusted Th1/Th2 ratio is plotted on the Y axis and the Th1/Th2 ratio obtained by the assay is plotted on the X axis. Ideally, each point should be plotted on the Y=X line, although deviations occur in practice. Thus, these plots are subjected to curve fitting using the method of least squares and the like to obtain a calibration curve. When curve fitting is carried out using a linear function, for example, a calibration curve for Y=pX+q is presumably obtained. p and q are parameters obtained by fitting. Specifically, formula 4-5 can be described as formula 4-5′ below.

$$Th1 / Th2 = {( \sum }_{n = 1}^{k} Th1 ( n ) / {\sum }_{n = 1}^{k} Th2 ( n ) ) \times p + q \qquad ( formula 4 \text{-} {5}^{′} )$$

When the calibration curve obtained by fitting is a quadratic function (Y=pX²+qX+r), a cubic function (Y=pX³+qX²+rX+s), or other nonlinear function, formula 4-5 can be similarly modified and used.

When the gene “n” is Th cell-specific and is expressed specifically in Th1 cells or in Th2 cells, the following formula 4-6 is used.

Th1/Th2=median (Rank(X₁), . . . , Rank(Xₙ₁))/median (Rank(Y₁), . . . , Rank(Yₙ₁))×β2 (formula 4-6)

Rank(X₁) is a ranking of the fluorescence intensity of a probe corresponding to a gene that is Th1 cell-specific, Rank(Y₁) is a ranking of the fluorescence intensity of a probe corresponding to a gene that is Th2 cell-specific, median is a medium value, and β2 is a normalizing parameter similar as with β in formula 4-5. Formula 4-6 is hereafter described.

It is hypothesized that there are n1 number of probes corresponding to Th1 cell-specific genes and that fluorescence intensities obtained from these probes are respectively X₁, X₂, . . . Xₙ₁. Also, there are n2 number of probes corresponding to the Th2 cell-specific genes and fluorescence intensities obtained from these probes are respectively Y₁, Y₂, . . . Yₙ₂. Ideally, the fluorescence intensity obtained from the Th2 cell sample by the probe corresponding to the Th1 cell-specific gene is zero, after background noise is subtracted. On the contrary, the fluorescence intensity obtained from the Th1 cell sample by the probe corresponding to the Th2 cell-specific gene is zero. Thus, the fluorescence intensity obtained by the probe corresponding to the Th1 cell-specific gene is only derived from the Th1 cell sample, and the fluorescence intensity obtained by the probe corresponding to the Th2 cell-specific gene is only derived from the Th2 cell sample. Comparison of the averages (or medians) among X₁, X₂, . . . Xₙ₁ and those among Y₁, Y₂, . . . Yₙ₂, however, cannot accurately determine Th1/Th2. This is because the Th1 cell-specific gene is different from the Th2 cell-specific gene, therefore, they cannot be simply compared to each other. If a sufficiently large number of probes are present on the chip and if probes are continuously present from those with higher fluorescence intensity to those with lower fluorescence intensity, when fluorescence intensities are rearranged in descending or ascending order for probes with higher or lower fluorescence intensities, genes with equivalent rankings are expected to be expressed at substantially the same levels among various genes in the cell sample. When all the probes on the DNA chip are rearranged in descending order by fluorescence intensity, the order of X₁, X₂, . . . Xₙ₁ is designated as Rank(X₁), Rank (X₂), . . . Rank(Xₙ₁), and the order of Y₁, Y₂, . . . Yₙ₁ is designated as Rank(Y₁), Rank(Y₂), . . . Rank(Yₙ₁). Since there are a plurality of Th1 specific genes and a plurality of Th2 specific genes, they are respectively represented by their medians. For example, the median (Rank(X₁), . . . Rank(Xₙ₁)):median (Rank(Y₁), . . . Rank(Yₙ₁)) ratio can be assigned as Th1/Th2. This median Rank ratio can be more accurate than simply using fluorescence intensity particularly when the Th1/Th2 ratio is close to 1. It should be noted that when the Th1/Th2 ratio is significantly different from 1, an accurate ratio may not always be reflected.

A statistical technique utilizing Bayesian estimation is described as follows. One type of RNA is previously divided into two, one of them is labelled with Cy3 while the other is labelled with Cy5. An experiment in which they are subjected to competitive hybridization on the same chip (approximately 5 to 10 chips are required) is hypothesized. This experimental data is equivalent to the observed value y=(y1, y2, . . . , yi, . . . , yn) obtained by extracting random samples of size n [Y1, Y2, . . . , Yi, . . . , Yn] from a normal population N(c, σ²) in which c=the average of Cy5/Cy3 is 1.0 while the variance σ² is unknown. Since error factors such as differences between chips, differences between coloring agents, and individual differences in handling are not correlated between experiments, [Y1, Y2, . . . , Yi, . . . , Yn] can be said to be mutually independently and identically distributed (i.i.d.).

Yi˜i.i.d.N(c, σ²)=N(1, σ²) (formula 5)

The ultimate goal of this Bayesian estimation is to estimate the σ² for Yi. If σ² for the population N(1, σ²) is estimated by this estimation, σ² for Yi is identical according to formula 5. In the Bayesian estimation, σ² is presumed to have a distribution. Thus, what is obtained is an estimate for σ². An average, a mode (a point of maximal frequency), or a Highest Density Region (HDR) as an confidence interval can be obtained as the estimate. The 90% highest density region is so to speak the shortest in the 90% interval of unknown parameter σ², and it always contains a peak value of posterior distribution (posterior mode). In addition, posterior densities at both ends of the interval are equal to each other.

When formula 5 is realized, the joint probabilistic distribution of [Y1, Y2, . . . , Yi, . . . , Yn] (the probabilistic distribution which simultaneously fulfills a1<Y1≦b1, a2<Y2≦b2, . . . , an<Yn≦bn) p(y′|c, σ2) can be represented in a form that is multiplied by a gaussian distribution:

$$p ( {y}^{′} \vert c , {σ}^{2} ) = {\prod }_{i = 1}^{n} ( 1 / \sqrt{} 2 π σ ) exp ( - 1 / 2 {σ}^{2} \times {( yi - c )}^{2} ) \qquad ( formula 6 )$$

wherein Π is a multiplication sign. Accordingly, when a vector of observed value y=(y1, y2, . . . , yi, . . . , yn) is provided, the likelihood function 1 (σ²|y) is either

$$1 ( {σ}^{2} \vert y ) ∝ {\prod }_{i = 1}^{n} ( 1 / σ ) exp ( - 1 / 2 {σ}^{2} \times {( y - c )}^{2} ) \qquad ( formula 7 )$$

or

1(σ²|y)∝(σ²)−n/2×exp(−ns²/2σ²) (formula 8).

It should be noted that s² is the distribution of observed values having the population mean c as a center and represented by the following formula.

$${s}^{2} = ( 1 / n ) \times {\sum }_{i = 1}^{n} {( yi - c )}^{2} \qquad ( formula 9 )$$

Noninformative prior distribution is hypothesized as the prior distribution p(σ²) of a variance. This hypothesis is reasonable in that the arbitrariness for unknown parameters of prior distribution is eliminated as much as possible, and posterior distribution is controlled by data as much as possible. In general, locally uniform prior distribution is employed as noninformative prior distribution. In locally uniform distributions, unknown parameters are uniformly distributed at least locally either in the form of squared, cubed, or logarithmic values in order to represent the vagueness of prior information. Specifically, it may be set so as to be in proportion with the square root of the Fisher information. If the locally uniform distribution is employed as prior distribution, it may be set as

p(σ²)∝σ⁻² (formula 10).

That is, the prior distribution p(σ²) of σ² is σ⁻², i.e., a constant. Posterior distribution is considered as follows. Based on the Bayesian theory,

p(σ²|y)∝1(σ²|y)p(σ²) (formula 11)

is effected. Thus, the posterior distribution p(σ²|y) of σ² is equal to χ⁻²(n, ns²) based on

p(σ²|y)∝(σ²)−(n/2+1)×exp(−ns²/2σ²) (formula 12).

χ²(ν, λ) is referred to as the inverse Chi square distribution with a scale parameter of λ and with ν degree of freedom. Since it is known that the average of χ⁻²(ν, λ) is λ(ν−2) and the mode (a point of maximal frequency) is λ/(ν+2), the following formulae can be considered as point estimates of σ²:

in the case of employing the average as the standard:

σ²=ns²/(n−2) (formula 13)

in the case of employing the mode as the standard:

σ²=ns²/(n+2) (formula 14).

Based on formula 12, the correlation

ns²/σ²˜χ²(n) (formula 15)

can be posteriorly obtained. χ²(n) of formula 15 is the Chi square distribution with n degrees of freedom. ns² is a fixed value (observed value), and σ² is a random variable. HDR can be obtained by using formula 15 and a mathematical table. When the Th1/Th2 ratio is obtained by using formulae 13, 14, and 15, the degree of statistically significant differences can be assessed by comparing these obtained values against 1.0 (Th1/Th2 value of a control).

For example, if y₁=1.4, y₂=0.89, y₃=1.24, y₄=0.91, and y₅=1.04 were obtained in 5 experiments, s²=0.04788. According to point estimation (formula 13) on the basis of the averages, Yi˜N(1, 0.0798) is obtained. According to point estimation (formula 14) on the basis of the modes, Yi˜N(1, 0.0342) is obtained. According to formula 15, σ²˜0.2394χ⁻²(5) is obtained. Thus, 90% HDR of σ² is found to be 0.019 to 0.177 by using a mathematical table.

Bayesian estimation represented by formula 5 to formula 15 is one statistical technique. Accordingly, methods other than Bayesian estimation, for example, Neyman-Pearson's estimation may be employed.

With the use of this Bayesian estimation, for example, when the Th cells ratio in all leukocytes is previously fixed at X (1.0 in the example above) and then the observed Th cells ratio is Y, the degree of statistically significant differences in the Th cells ratio can be computed. The ratios of T cells, B cells, and the like in all leucocytes are also valuable in terms of assessing the subject's physical condition. If there is any significant change in the Th cells ratios in all leucocytes, the subject can be diagnosed as previously having had some changes in his/her physical condition.

### EXAMPLES

One example of the present invention, which simulates an example for assessing changes in the subject's allergy level, is described. Since it is difficult to obtain blood from an allergy patient, Th1 cells, Th2 cells, and monocytes are separated and extracted from peripheral blood of a normal volunteer, and the mixing ratios of these cells are varied to simulate the examination using whole blood. Monocytes express many genes similar to Th cells, and thus, there present a major bias in the assay using whole blood. If the Th1 cells:Th2 cells ratio can be accurately determined even when monocytes are present, the bias derived from the existence of B cells and the like can be similarly eliminated and the assay can be accurately carried out even when whole blood is assayed.

The observed Th1/Th2 values are described, (1) when a sample containing only Th1 and Th2 cells is assayed and (2) when a sample containing monocytes besides Th1 and Th2 cells is assayed. The mixing ratios of Th1 cells and Th2 cells were previously set at 1:9, 2:8, 3:7, 4:6, 5:5, 6:4, 7:3, 8:2, and 9:1.

Th1/Th2 is represented by formula 1 or formula 2. In the examples herein, the definition of formula 1 is used when preparing samples. The definition of formula 2 is used for observed values. In general, formula 1 and formula 2 are in agreement when the gene expression level per cell does not vary between each subject of the examination. Formula 1 and formula 2, however, are not always in agreement when, for example, Th cells previously had genetic abnormalities and the expression level of a certain cytokine differs from a normal level. In this specification, formula 1 and formula 2 were substantially in agreement as a result of the use of the blood of a normal volunteer, however, the results on a normal volunteer as such cannot be always applicable for some specific diseases. In these cases, the present invention may be applied for each disease after formula 1 and formula 2 are independently evaluated.

The expression levels of the genes that are expressed specifically in monocytes, correspond to those of the genes that are not substantially expressed in Th cells but are expressed specifically in cells other than T cells, such as B cells or monocytes. For example, the abundance ratio of Th cells to B cells can be similarly determined as formula 3.

In the description given hereinafter, as the expression levels of genes that are expressed specifically in Th cells, the average fluorescence intensities obtained from a plurality of genes that are expressed specifically in Th cells is employed. Similarly, the average fluorescence intensities obtained from a plurality of genes that are expressed specifically in monocytes is employed as the expression level for genes that are expressed specifically in monocytes. The average fluorescence intensities obtained from a plurality of genes that are expressed equally in both Th cells and monocytes is employed as the expression level for genes that are expressed equally in both Th cells and monocytes.

When assaying Th1/Th2 based on the assay result using DNA chips, a method can be utilized in which only the genes that are expressed differently in Th1 cells and in Th2 cells, and are expressed specifically in Th cells are used. Genes that are expressed specifically in Th cells are, for example, CD2, CD28, CD3D, CD3E, CD3G, CD3Z, CD8A, CTLA4, MIC2, SEMA4D, THY1, TRA@, and TRB@ (Symbol designations of the Unigene). However, since only a small number of genes fall under this range, there were relatively large variations in the observed values as a result of our data analysis. In this example, therefore, Th1/Th2 was determined by formula 4.

As annotation of the human genome progresses, in the future, it can be expected that the number of genes, which belong to genes that are expressed differently in Th1 cells and in Th2 cells and are expressed specifically in Th cells, would increase to the extent that assay variation can be sufficiently lowered, for example 50 to 200. In that case, the use of genes that are expressed differently in Th1 cells and in Th2 cells and are expressed specifically in Th cells may result in achieving sufficient assay sensitivity and reproducibility. In such cases, only formula 4-1 and formula 4-2 may be used. The experimentation methods are described as follows:

Formulae 1 to 4 in this specification were computed by using all the genes as shown in Tables 1 to 9.

1. Acquisition of Th1 Cells and Th2 Cells from Blood

Whole blood (50 ml) was sampled from a normal volunteer with a heparin-coated hypodermic needle. Serum free RPMI 1640 medium (supplemented with 1% penicillin/streptomycin) (50 ml) was added to the whole blood and diluted. This diluent was poured into a 50 ml centrifuge tube containing 10 ml of Ficoll-Paque, and centrifugation was carried out at room temperature at 800 g for 30 minutes. Peripheral blood mononuclear cells (PBMC) were carefully sampled from the interface and washed twice with Phosphate Buffered Saline (PBS). Monocytes were removed by depositing onto a gelatine coated tissue culture flask. RPMI 1640 medium+10% FCS+5 μg/ml PHA was added to PBMC. IL 12 (1 ng/ml) and anti-IL4 antibody (0.1 μg by diluting 500 μg to 5,000-fold) were added to the PBMC medium mentioned above, and Th1 cells were cultured therein. Medium was changed every 3 days. IL4 (50 ng/ml) and anti-IL12 antibody (0.1 μg by diluting 500 μg to 5,000-fold) were added to the PBMC medium mentioned above, and Th2 cells were cultured therein. Medium was changed every 3 days. Under these culture conditions, approximately two thirds of the cells were CD4+ cells and the remaining one third was CD8+ cells. Monocytes, B cells, and NK cells were not substantially present. In order to remove CD8+ cells, anti-CD8+ monoclonal antibodies were added, and incubation was carried out at 4° C. for 30 minutes. Thereafter, unbound monoclonal antibodies were removed and the remaining mixture was bound to magnetic beads whose surfaces had been coated with a goat anti-mouse Fc-specific antibody. The ratio of the number of beads:the number of cells was 25:1. CD8+ cells were removed by a bead trap using magnetic force, and the residual CD8+ ratio was not more than 1%. The thus obtained Th1 cells and Th2 cells were immediately used or cryopreserved in liquid nitrogen. The composition of the preserved solution was 95% medium/5% DMSO.

2. Acquisition of Monocytes from Blood

Whole blood (50 ml) was sampled from a normal volunteer with a heparin-coated hypodermnic needle. Serum free RPMI 1640 medium (supplemented with 1% penicillin/streptomycin) (50 ml) was added to the whole blood and diluted. This diluent was poured into a 50 ml centrifuge tube containing 10 ml of Ficoll-Paque, and centrifugation was carried out at room temperature at 800 g for 30 minutes. PBMC was carefully sampled from the interface and washed twice with PBS. Thereafter, RPMI 1640 medium+7.5% FCS+100 μg/ml streptomycin and 100 U/ml penicillin were added to the PBMC culture. PBMC was mixed with magnetic beads whose surfaces had been coated with anti-CD14 monoclonal antibody, and the resulting mixture was incubated and separated by a bead trap using magnetic force. The separated monocytes were cultured at 37° C. in a 5% CO₂ incubator for 30 minutes, and high purity monocytes were obtained. Positive staining using CD14 and flow cytometry were carried out. As a result, 99% or more were confirmed to be monocytes. The thus obtained monocytes were immediately used or cryopreserved in liquid nitrogen. The composition of the preserved solution was 90% medium/10% DMSO.

3. Acquisition of Total RNA from Th Cells and Monocytes and Preparation of RNA Mixture

Total RNA was obtained from the cells using ISOGEN (Nippon Gene Co., Ltd.). Specifically, 750 ml of ISOGEN was added to 5×10⁶ Th1 cells, Th2 cells, or monocytes, respectively. This process is the process in which a cell membrane is disrupted to collect nucleic acid from the cell and phenol extraction is performed. After centrifugation was carried out at 4° C. at 15,000 rpm for 30 minutes, the supernatant was carefully collected with a pipette and transferred to a new Eppendorf tube. Thereafter, the following were carried out: chloroform extraction twice, isopropyl precipitation once, and rinsing with 70% ethanol once. The RNA, which remained at the bottom of the Eppendorf tube, was dissolved in TE buffer. The RNA concentration was determined by measuring absorption. OD260/OD280 was 1.9 to 2.0. The thus obtained Th1 cell-derived total RNA and Th2 cell-derived total RNA were mixed with each other at 1:9, 2:8, 3:7, 4:6, 5:5, 6:4, 7:3, 8:2, and 9:1 based on the weight ratio of RNA. In the case where monocytes were added, the abundance ratio of T cells to monocytes in leukocytes were taken into consideration to bring the weight ratio of RNA derived from both cells to Th1 cells or Th2 cells:monocytes=5:4. This ratio is equivalent to the ratio of 5×10⁸, which is half the number of T cells, 1×10⁹, in 1 liter of human blood, and 4×10⁸, which is the number of monocytes and/or macrophages in 1 liter of human blood.

4. Preparation of DNA Array

Subsequently, 793 oligonucleotide probes, which have high specificity and identical Tm, were designed in accordance with the algorithm comprising the steps of:

1. reading a gene sequence file;

2. inputting experimental conditions on, for example, salt concentration and hybridization;

3. inputting a length range of DNA fragments to be immobilized;

4. calculating the melting temperature of each candidate DNA fragment for immobilization and eliminating from the candidate list the DNA fragments whose melting temperatures are outside a given range;

5. eliminating from the candidate list DNA fragments, which have a short sequence having specific higher order structure or short repeated sequence;

6. eliminating from the candidate list DNA fragments, which are highly homologous to repeated sequences such as the Alu sequence;

7. eliminating from the candidate list DNA fragments, which are highly homologous to other gene sequences;

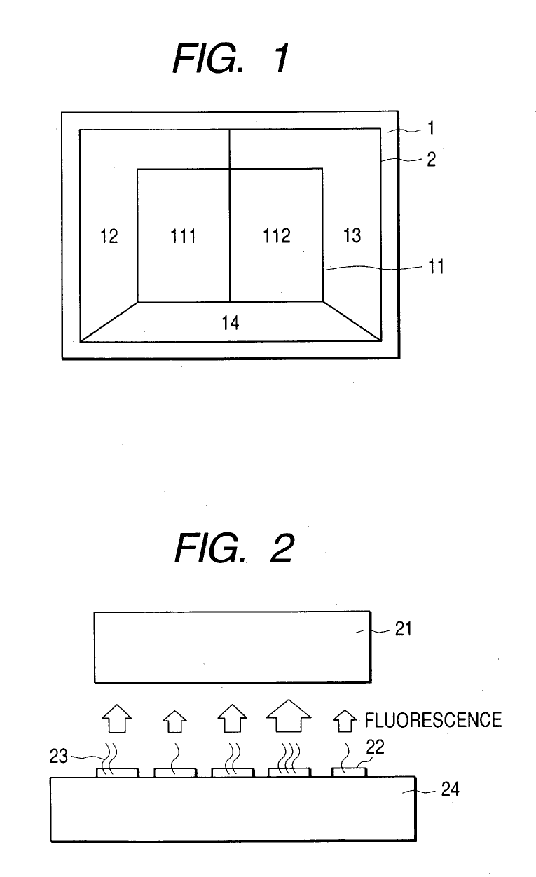

and the like. In addition to these 793 types of human gene probes, 3 types of oligonucleotide sequences (lambda DNA, pUC18 plasmid DNA, and M13mp18 DNA), which are absent in humans, were added as external control genes for proofreading to prepare 796 types of oligonucleotides, and these were immobilized on a glass substrate in the manner as disclosed below. When immobilizing on the glass substrate, DNA fragments were positioned, for example, as shown in FIG. 1. In FIG. 1, the following are separately immobilized: probe DNA of genes that are expressed specifically in Th cells (12), probe DNA of genes that are not expressed very much in Th cells but are expressed specifically in cells other than T cells, such as B cells and monocytes (13), probe DNA of genes that are expressed equally in Th cells and in other cells (14), and DNA fragments (probe DNA) having gene sequences that are expressed differently in Th1 cells and in Th2 cells or complementary sequence thereof (11). Further, the following are separately immobilized: probe DNA (111) of genes that are expressed differently in Th1 cells and in Th2 cells and whose expression level in Th1 cells is higher than that in Th2 cells, and probe DNA (112) of genes that are expressed differently in Th1 cells and in Th2 cells and whose expression level in Th2 cells is higher than that in Th1 cells.

Prior to the immobilization of the probes, a commercially available slide glass (Gold Seal Brand) was first immersed in an alkaline solution for 2 hours at room temperature. Thereafter, the slide glass was transferred into distilled water and rinsed 3 times to completely remove the alkaline solution. Subsequently, the rinsed slide glass was immersed in an aqueous solution of 10% poly-L-lysine (Sigma) for 1 hour, the slide glass was then pulled out, and the aqueous solution of poly-L-lysine was removed by centrifugation for 1 minute by using a centrifuge for micro titer plates. Thereafter, the slide glass was put into a vacuum incubator and dried at 40° C. for 5 minutes, to introduce an amino group on the slide glass.

An automatic DNA synthesizer was used to synthesize oligonucleotides, and the oligonucleotides were then purified by High-Performance Liquid Chromatography. Subsequently, the 1 μl of synthesized and purified oligonucleotide (concentration of 2 μM), additives, and the like were mixed to prepare a spotting solution. The prepared spotting solution was spotted on the slide glass at desired positions using a spotter. FIG. 2 shows a general structure of a DNA chip. As shown in FIG. 2, fluorescence-labelled gene 23 hybridizes with DNA probe 22 immobilized on support 24 in a sequence-specific manner, and the fluorescence emitted from the bound label is detected by fluorescence detector 21.

5. Assay Result

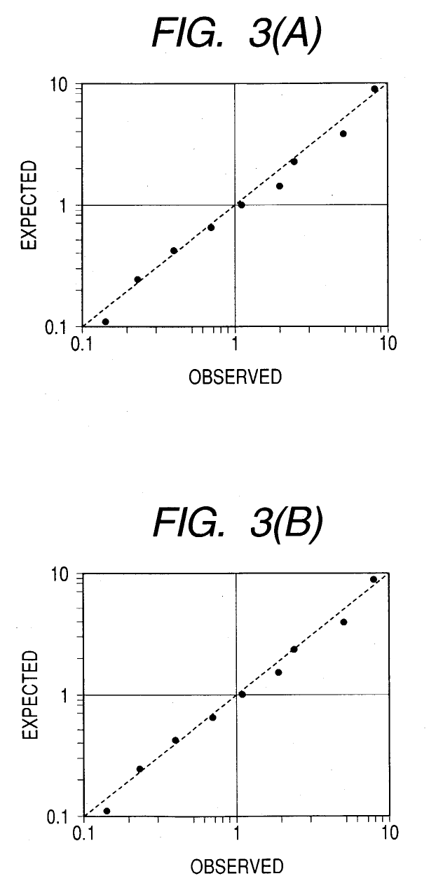

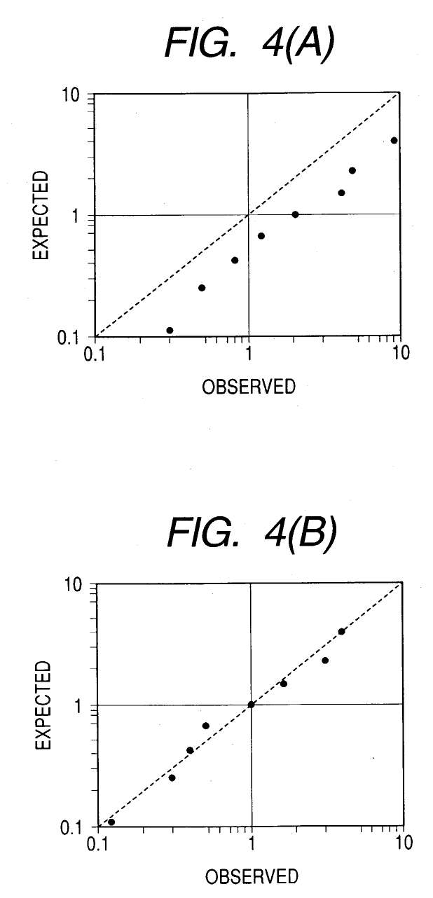

FIG. 3 shows an example in which a sample containing only Th1 and Th2 cells is assayed to evaluate Th1/Th2, and FIG. 4 shows an example in which a sample containing monocytes in addition to Th1 and Th2 cells is assayed to evaluate Th1/Th2. FIG. 3(A) is a result obtained when Th1/Th2 was evaluated by using only genes that are expressed differently in Th1 cells and in Th2 cells. FIG. 3(B) is a result obtained, using formula 4 (formulae 4-1 to 4-6), when the following genes were used: genes that are expressed differently in Th1 cells and in Th2 cells, genes that are expressed specifically in Th cells, genes that are not expressed very much in Th cells but are expressed specifically in cells other than T cells, such as B cells and monocytes, and genes that are expressed equally in Th cells and in other cells. FIG. 4(A) and FIG. 4(B) are the similar as FIG. 3. In the graphs of FIG. 3 and FIG. 4, the X axis indicates the observed values and the Y axis indicates the expected values. The expected values represent Th1/Th2 (formula 1) determined from the mixing ratio of Th1 cells and Th2 cells, and the observed values represent Th1/Th2 (formula 2) determined from the gene expression level. As shown in both (A) and (B) in FIG. 3, Th1/Th2 was substantially equally assayed from the sample containing only Th1 and Th2 cells. Tis is because α/(α+1) in formula 4 is almost 1. As shown in FIG. 4(A), however, if the sample contains cells other than Th cells, in this case, monocytes, Th1/Th2 cannot be accurately assayed and assay variation is also large. This is because α/(α+1) is not equal to 1. In this example, Th1 cells or Th2 cells:monocytes=5:4, i.e, α=5/4, and thus, α/(α+1)=0.555. The assignment of this value 0.555 into formula 4-5 and the use of the normalizing parameter β=0.92 separately obtained when Th1:Th2 is 1:1 enables the substantially accurate assay of Th1/Th2 as shown in FIG. 4(B). This can be realized because contribution of monocytes-derived gene expression is corrected by formula 4. Thus, Th1/Th2 can be assayed without the isolation of Th cells. This process enables the experiments to be carried out in a simpler manner, at lower cost, and more rapidly.

In the present invention, changes in the expression levels of: (i) genes that are expressed differently in Th1 cells and in Th2 cells; as well as (ii) genes that are expressed specifically in Th cells; (iii) genes that are not expressed in Th cells; and (iv) genes that are expressed equally in Th cells and in other cells, were assessed, and in particular, the value of (i) was corrected by using values of (ii), (iii), and (iv). This improved the assay accuracy, and has led to the completion of the present invention.

Th1 cells and Th2 cells are associated with various immune disorders. For example, Th1 cells are known to significantly infiltrate into synovial fluids of patients afflicted with rheumatoid arthritis, and Th2 cells are known to selectively infiltrate into skins of patients afflicted with atopic dermatitis. Compared to a normal volunteer, observed in the blood of patients with atopic dermatitis are an increase in the number of Th2 cells which express CCR4 and a decrease in the number of Th1 cells which express CXCR3, (Murai and Matsushima, Saibou Kougaku (Cell Technology), Vol. 19, 703-707, 2000). In addition, strong association between Th1/Th2 imbalance and asthma, AIDS, tuberculosis, Lyme disease, type I diabetes, rheumatoid arthritis, multiple sclerosis, and the like have been pointed out. It is expected that controlling the Th1/Th2 balance can prevent and treat these diseases.

There are several cytokines generated in Th1 cells, for example, IFN-gamma and IL2. Also, there are several cytokines generated in Th2 cells, for example, IL4, IL5, IL6, IL9, IL10, and IL13. Among these several cytokines, the IFN-gamma: IL4 ratio is said to be the closest to Th1 cells:Th2 cells ratio. There are, however, quite a number of immune disorders associated with normal IFN-gamma:IL4 ratios. For example, in idiopathic nephrotic syndrome, which is presumed to be deeply associated with atopic dermatitis, there is no difference in the IFN-gamma mRNA:IL4 mRNA ratio in the peripheral blood cell between patients and normal volunteers. However, the level of IL13 mRNA in the peripheral blood mononuclear cell is significantly increased in patients (Yap, H-K et al., Th1 and Th2 cytokine mRNA profiles in childhood nephrotic syndrome: Evidence for increased IL-14 mRNA expression in relapse, J. Am. Soc. Nephrol 10, 529-537, 1999). This indicates that even though there is no difference between the number of Th1 cells and the number of Th2 cells, the level of produced cytokines is imbalanced if either Th1 cells or Th2 cells have abnormalities. Since the DNA microarray enables the observation of a large number of genes associated with the Th1/Th2 balance at the same time, it can deal with various immune disorders and a cost required for one diagnosis can be lowered.

### EFFECT OF THE INVENTION

The present invention provides a method and a system for allergy analysis by using RNA derived from leukocytes extracted from peripheral blood as such, correcting, assessing the balance in helper T (Th) cells (Th1 cells:Th2 cells ratio-(Th1/Th2)), and comparing the balance with the number of Th1 cells/the number of Th2 cells in a nucleic acid sample or the Th1/Th2 data of a patient and of a normal volunteer. The present invention can also provide a method and a system for allergy analysis using a highly reproducible and reliable array having a minimal number of DNA fragments (oligonucleotides) thereon, which is realized by identifying essential gene groups for the Th1/Th2 assay.

All publications, patents and patent applications cited herein are incorporated herein by reference in their entirety.

What is claimed is:

1. A method for allergy analysis based on the balance of Th1 cells to Th2 cells ratio (Th1/Th2), comprising the steps of:
labeling a nucleic acid sample derived from human peripheral blood with fluorescence;
performing hybridization of the nucleic acid sample labeled with fluorescence with a plurality of probes, wherein the plurality of probes correlate with genes which contain genes that are expressed specifically in Th cells, genes that are expressed specifically in Th1 cells, or genes that are expressed specifically in Th2 cells, or fragments thereof;
detecting the presence or absence of hybridization by the fluorescence as the expression level;
assaying the expression levels of one or more genes that are expressed specifically in Th1 cells and in Th2 cells respectively based on detected fluorescence intensities; and
comparing (the number of Th1 cells)/(the number of Th2 cells) in the nucleic acid sample or the Th1/Th2 data of a patient and of a normal volunteer with (the expression levels of one or more genes that are expressed specifically in Th1 cells)/(the expression levels of one or more genes that are expressed specifically in Th2 cells) as Th1/Th2.

2. The method for allergy analysis according to claim 1, wherein when the gene which correlate with the plurality of probes further contain genes that are expressed specifically in cells other than Th cells and the nucleic acid sample is also expressed in cells other than Th cells, as the expression level for one or more genes that are expressed specifically in Th1 cells and that in Th2 cells, the value of (fluorescence intensity)×(α/(α+1)) (α: the expression level for one or more genes that are expressed specifically in Th cells/the expression level for one or more genes that are expressed specifically a cell other than Th cells) is employed, respectively.

3. The method for allergy analysis according to claim 2, wherein as the expression level for one or more genes that are expressed specifically in Th1 cells and that in Th2 cells, a sum or an average of fluorescence intensities is employed, respectively, and the product with β, a normalizing function to realize Th1/Th2=1 when Th1:Th2 is 1:1, is used as the value of Th1/Th2.

4. The method for allergy analysis according to claim 2, wherein as the expression level for one or more genes that are expressed specifically in Th1 cells and that in Th2 cells, a median of the fluorescence intensity ranking among the plurality of probes is employed, respectively, and the product with β is used as the value of Th1/Th2.

5. The method for allergy analysis according to claim 2, wherein the cell other than Th cells is at least one type of cells selected from neutrophils, eosinophils, basophils, monocytes, macrophages, B cells, and NK cells.

6. A system for allergy analysis based on the balance of Th1 cells to Th2 cells ratio (Th1/Th2) comprising:
an oligonucleotide array having a plurality of probes immobilized thereon, wherein the plurality of probes correlate with genes comprising genes that are expressed specifically in Th cells, genes that are expressed specifically in Th1 cells, and genes that are expressed specifically in Th2 cells;
a detection means for detecting, as the expression level, the fluorescence of a nucleic acid sample that is hybridized with the probes;
a data storage means for storing data on the number of Th1 cells and the number of Th2 cells in the nucleic acid sample or Th1/Th2 for a patient and for a normal volunteer;
a data storage means for storing data on the expression level for one or more genes that are expressed specifically in Th1 cells and in Th2 cells based on detected fluorescence intensities; and
a computer for comparing (the number of Th1 cells)/(the number of Th2 cells) in the nucleic acid sample or the Th1/Th2 data of a patient and of a normal volunteer with the (expression level for one or more genes that are expressed in Th1 cells)/(the expression level for one or more genes that are expressed in Th2 cells) as Th1/Th2.

7. The system for allergy analysis according to claim 6, wherein when the gene which correlate with the plurality of probes further contain genes that are expressed specifically in cells other than Th cells and the nucleic acid sample is also expressed in cells other than Th cells, as the expression level for one or more genes that are expressed specifically in Th1 cells and that in Th2 cells, the value of (fluorescence intensity)×(α/(α+1)) (α: the expression level for one or more genes that are expressed specifically in Th cells/the expression level for one or more genes that are expressed specifically in cells other than Th cells) is employed, respectively.

8. The system for allergy analysis according to claim 7, wherein as the expression level for one or more genes that are expressed specifically in Th1 cells and that in Th2 cells, a sum or an average of the fluorescence intensities is employed, respectively, and the product with β, a normalizing function to realize Th1/Th2=1 when Th1:Th2 is 1:1, is used as the value of Th1/Th2.

9. The system for allergy analysis according to claim 7, wherein as the expression level for one or more genes that are expressed specifically in Th1 cells and that in Th2 cells, a median of the fluorescence intensity ranking among the plurality of probes is employed, respectively, and the product with β is used as the value of Th1/Th2.

10. The system for allergy analysis according to claim 7, wherein the cell other than Th cells is at least one type of cells selected from neutrophils, eosinophils, basophils, monocytes, macrophages, B cells, and NK cells.
